(19) United States
(12) Patent Application Publication
(10) Pub. No.: US 2025/0308648 A1
(43) Pub. Date: Oct. 2, 2025

(54) A METHOD, AN APPARATUS AND A COMPUTER PROGRAM PRODUCT FOR EVALUATING COGNITIVE PERFORMANCE OF A USER

(71) Applicant: Wellpro Impact Solutions Oy, Jyväskylä (FI)

(72) Inventors: Petteri SVEINS, Veikkola (FI); Juha TENHUNEN, Jyväskylä (FI); Juho STRÖMMER, Jyväskylä (FI); Joni BORGSTRÖM, Jyväskylä (FI)

(73) Assignee: Wellpro Impact Solutions Oy, Jyväskylä (FI)

(21) Appl. No.: 18/864,054

(22) PCT Filed: May 9, 2022
(86) PCT No.: PCT/FI2022/050310

§ 371 Date: Nov. 8, 2024

(51) Int. Cl. G16H 10/20

(52) U.S. Cl. CPC G16H 10/20

(57) ABSTRACT

The embodiments relate to screening solution that is used for evaluation peoples' cognitive performance. The solution is based on a method including receiving data concerning user's personal information; determining at least one group of questions based on the received data, wherein the group of questions has psychological questions relating to cognitive performance of a user; determining weights for each question in the at least one group of questions based on the personal information; providing the at least one group of questions to a user; receiving an input from the user, the input having answers to the group of questions; and determining an evaluation index based on the answers received to all questions of the at least one group for questions, the evaluation index indicating user's cognitive performance.

### TECHNICAL FIELD

The present solution generally relates to a solution for evaluating cognitive performance of user.

### BACKGROUND

Increasingly more senior citizens are suffering from memory diseases and memory disorders. In addition to seniors, also middle-aged people may be touched by memory disorders. If memory disorders and memory diseases are diagnosed early, this will positively affect to patient's performance in the future. Maintaining the nearly normal living with the lowered cognitive performance may alleviate the symptoms of the memory disorder, and may also postpone contracting the actual memory disease (e.g. dementia).

### SUMMARY

The purpose of the present solution is to provide a method and technical equipment by means of which people's cognitive performance can be easily evaluated. The solution is applicable for screening, whereupon cognitive performance of mass of people can be simply assessed.

The scope of protection sought for various embodiments of the invention is set out by the independent claims. The embodiments and features, if any, described in this specification that do not fall under the scope of the independent claims are to be interpreted as examples useful for understanding various embodiments of the invention.

Various aspects include a method, an apparatus and a computer readable medium comprising a computer program stored therein, which are characterized by what is stated in the independent claims. Various embodiments are disclosed in the dependent claims.

According to a first aspect, there is provided a method, comprising receiving data concerning user's personal information; determining at least one group of questions based on the received data, wherein the group of questions comprises psychological questions relating to cognitive performance of a user; determining weights for each question in said at least one group of questions based on the personal information; providing said at least one group of questions to a user; receiving an input from the user, said input comprises answers to the group of questions; and determining an evaluation index based on the answers received to all questions of said at least one group for questions, the evaluation index indicating user's cognitive performance.

According to a second aspect, there is provided an apparatus comprising means for receiving data concerning user's personal information; means for determining at least one group of questions based on the received data, wherein the group of questions comprises psychological questions relating to cognitive performance of a user; means for determining weights for each question in said at least one group of questions based on the personal information; means for providing said at least one group of questions to a user; means for receiving an input from the user, said input comprises answers to the group of questions; and means for determining an evaluation index based on the answers received to all questions of said at least one group for questions, the evaluation index indicating user's cognitive performance.

According to a third aspect, there is provided an apparatus comprising at least one processor, memory including computer program code, the memory and the computer program code configured to, with the at least one processor, cause the apparatus to perform at least the following:
receive data concerning user's personal information;
determine at least one group of questions based on the received data, wherein the group of questions comprises psychological questions relating to cognitive performance of a user;
determine weights for each question in said at least one group of questions based on the personal information;
provide said at least one group of questions to a user;
receive an input from the user, said input comprises answers to the group of questions; and
determine an evaluation index based on the answers received to all questions of said at least one group for questions, the evaluation index indicating user's cognitive performance.

According to a fourth aspect, there is provided computer program product comprising computer program code configured to, when executed on at least one processor, cause an apparatus or a system to:
receive data concerning user's personal information;
determine at least one group of questions based on the received data, wherein the group of questions comprises psychological questions relating to cognitive performance of a user;
determine weights for each question in said at least one group of questions based on the personal information;
provide said at least one group of questions to a user;
receive an input from the user, said input comprises answers to the group of questions; and
determine an evaluation index based on the answers received to all questions of said at least one group for questions, the evaluation index indicating user's cognitive performance.

According to an embodiment, a group-specific index is determined for each group of questions based on the answers received to such group, wherein the group-specific index takes into account the weights of each question.

According to an embodiment, the evaluation index is compared to a reference group and optionally also to previous evaluation index/indices of the user.

According to an embodiment, a feedback is generated based on the group-specific index/indices, the evaluation index and result of the comparison.

According to an embodiment, the computer program product is embodied on a non-transitory computer readable medium.

### DESCRIPTION OF THE DRAWINGS

In the following, various embodiments will be described in more detail with reference to the appended drawings, in which

FIG. 1 shows an example of a method;

FIG. 2 shows an overview of various elements implementing the method;

FIG. 3 shows an example of a neural network according to an embodiment;

FIG. 4 shows processing modules according to an embodiment;

FIG. 5 shows a data structure for a question according to an embodiment; and

FIGS. 6a, 6b show a simplified example on determining a suitable questions for a specific respondent.

### DESCRIPTION OF EXAMPLE EMBODIMENTS

The following description and drawings are illustrative and are not to be construed as unnecessarily limiting. The specific details are provided for a thorough understanding of the disclosure. However, in certain instances, well-known or conventional details are not described in order to avoid obscuring the description. References to one or an embodiment in the present disclosure can be, but not necessarily are, reference to the same embodiment and such references mean at least one of the embodiments.

Reference in this specification to “one embodiment” or “an embodiment” means that a particular feature, structure, or characteristic described in connection with the embodiment in included in at least one embodiment of the disclosure.

The aim of the present embodiments is to provide a solution for evaluating peoples' cognitive performance. The solution is based on a plurality of psychological questions that are provided to a respondent (also referred to as “a user”). The questions are related to various fields that have been identified to affect person's cognitive performance. The fields comprise mind and mental well-being; recovery and physical well-being; personal satisfaction to life and oneself; and social relationships and social well-being. The questions are provided to an electronic device of a user (i.e., a respondent), and displayed on a display of the electronic device. The respondent may answer the questions by using the electronic device. Each of the answers is evaluated by the system, and a result is determined. The result comprises at least one index that indicates person's cognitive performance. The system may be configured to generate a report on next operational steps based on said at least one index.

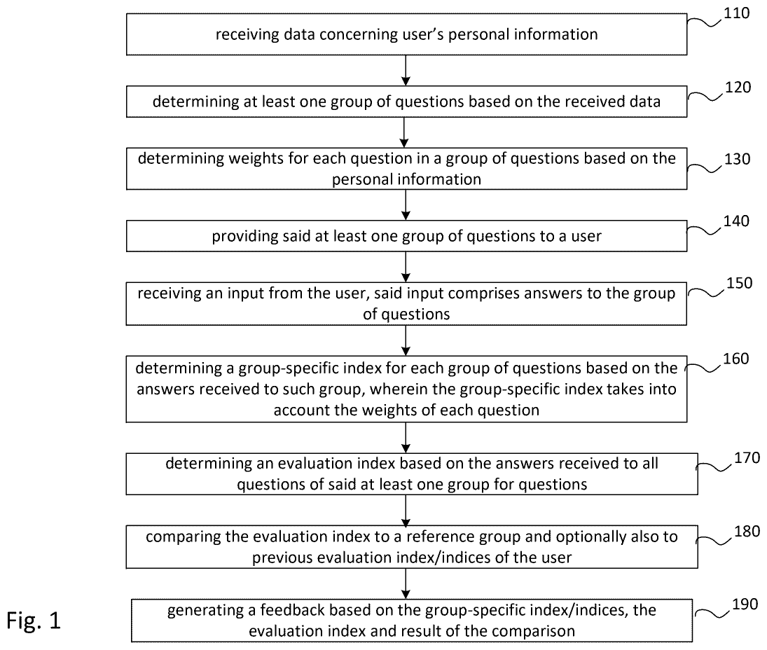

The solution is discussed in more detailed manner with a reference to FIG. 1. According to an embodiment, the method comprises one or more of the following steps:
receiving 110 data concerning user's personal information;
determining 120 at least one group of questions based on the personal information;
determining 130 weights for each question in said at least one group of questions based on the personal information;
providing 140 said at least one group of questions to a user;
receiving 150 input from the user, said input comprising answers to said at least one group of questions;
determining 160, for a user, a group-specific index for each group of questions based on answers received to such questions, wherein the group-specific index is determined by calculating an average of the answers of the group;
determining 170 an evaluation index based on the answers received to all questions of said at least one group of questions;
comparing 180 the evaluation index to a reference group and optionally also to previous evaluation index/indices of the user;
generating 190 feedback based at least on the evaluation index and alternatively also the result of the comparison.

It is appreciated that some of the steps can be ignored, for example, sometimes it may be enough to determine only one index, i.e., the evaluation index. Each of the steps can be implemented by a respective module of a computer system.

Personal information may be automatically obtained 110 when the user accesses the query and identifies himself/herself during access. Instead or in addition, some of the personal information may be provided by the user at the time the user access the service providing the query. For example, personal information may be provided from one or more third party services, e.g., the Population Registration Centre, health record centre, gene bank, but some of the personal information, e.g., the working status, the education, the marital status, the number of children may be provided by the user as background information.

Based on the respondent's personal information, the system is configured to select 120 questions to form a query for a user in question. If the user has answered the query before, also previous answers may be utilized to select the questions.

Questions of the query may have been classified into several specified groups according to their fields of interest. Thus, each question may be associated with an indication of a group the question belongs to. The grouping of the questions may or may not be indicated to the user. The groups can be for example “Mind”, “Movement”, “Me”, “Others”, wherein the questions falling into group “Mind” aim for solving the mental well-being of the user; the questions falling into group “Movement” aim for solving the physical well-being of the user; the questions falling into group “Me” aim for solving the personal well-being and satisfaction; the questions falling into “Others” aim for solving the user's social well-being. The questions are displayed to the respondent one by one, and question-related answer selection is provided alongside.

The selected sets (i.e., groups) of questions may be provided 140 to a user by conventional means, i.e., by displaying it on a user interface of an electronic device that is connected to the question database via wireless or wired network. The electronic device may be user's own device, for example a personal computer, a laptop computer, a tablet device or a smartphone. The questions may be displayed with a set of answers, from which a suitable answer may be selected by the respondent. The answers may be provided as multiple choice answers or as numerical selections. Instead or in addition, an empty field may be provided to a respondent, into which an answer as a free text may be input.

The input 150 from the user in the form of answers can be received by conventional means. For example, the user may use a keyboard, a mouse or any other pointer device (examples including also fingers of the user), voice commands, etc. to enter data and/or to make answer-related selections on the user interface.

As said, the answers may be pre-formatted as multiple choice answers or may be in numerical format. Alternatively or in addition, the answers may be open answers to be provided by the respondent as free text. When in a numerical format, the questions may be answered by giving a certain value from a range [1, 10] (inclusive). For example, number “1” may indicate that a question does not apply to a respondent; and a number “10” may indicate that a question concerns the respondent extensively.

Based on the answers received to the questions and the groups associated with the questions, a group-specific index may be calculated 160 for each group. These indices define user's well-being in corresponding groups.

The group-specific index may be determined by calculating an average of the answers for questions of the respective group. Prior the average calculation, each answer is given a weight of, e.g., 0.75-1.25 based on the personal information obtained. The weights have been predefined for the questions so that the system is able to determine (based on the previous answers and/or personal information) how the given answer for the specific question should be weighted. The weight range of 0.75-1.25 is given as an example, and can-in some other situations—be different. By default, a weight of each question in that weight range can be 1.00. However, for example, if it is determined from the personal information that the user has a physical disability, then questions relating to physical activity may be weighted lower, e.g., with value 0.75. As another example, if the personal information indicates that the user has a raised risk for hereditary memory disorder (e.g., dementia), then questions relating to cognitive performance may be weighted higher, e.g., with value 1.25.

Averaging is straightforward operation when the answers are given in numerical format or selected from multiple choices. If the answer is given as free text, an algorithm enabling artificial intelligence, may be used to identify keywords and converting the text into numerical value based on the keywords. These numerical values can then be used for determining the average.

As an example of the averaging, the following hypothetical calculation is given:
Questions of Group_1 receive answers 1, 4, 2, 7;
Questions of Group_2 receive answers 10, 10, 9, 10;
Questions of Group_3 receive answers 5, 6, 4, 8.

If the user's personal information reveals that the user is 20 years old, the weight for Group_2 is 0,75. If, on the other hand the user's personal information reveals that the user is 80 years old, the weight for Group_2 is 1,25. Instead of weighting all the questions falling within a certain group, single questions may be weighted as well.

With this information, the group-specific indices are the following:
Group_1_idx=3,5
Group_2_idx (20years)=7,3
Group_2_idx (80years)=12,2
Group_3_idx=5,8

As a result, the system gets as many group-specific indices as there are groups, wherein the group-specific indices indicate person's performance and/or well-being in that group.

In addition to or instead of the group-specific indices, an evaluation index is calculated 170. To determine the evaluation index, answers of all the questions (with weights) of the query are taken into account. Otherwise the index calculation follows the principles of the group-specific index calculation.

With the previous example, the evaluation index would then be
Ev_idx(20 years)=5,52
Ev_idx(80 years)=7,15

If the personal information comprises a great variety of information taken from third-party services, the system may further utilize such information when determining the evaluation index. For example, instead of only defining weights based on the personal information, some of the personal information may cause an additional value to be added to the average calculation. The determined evaluation index is an indication of overall cognitive performance of the user. The system may compare 180 the evaluation index to one or more reference groups. The reference group is formed of people having the same or approximately the same age, the same gender, the same disability, the same work situation, the same type of work, etc. or any same combination of them. In addition to the reference group, the evaluation index may be compared to the evaluation indices of the respondent having been determined earlier. The purpose of the latter comparison is to indicate any progress or regression from the past.

After the user's performance with respect to the reference group and/or to user's previous indices has been determined, a feedback report may be generated 190. The feedback report may be displayed on the display of the electronic device of the user, and additionally or instead, delivered to the user via an email, a conventional letter, etc. In addition, the report may be delivered to a healthcare provider if this has been agreed on.

The feedback report describes what is person's risk in the specific groups, as well as what is the overall risk when all the groups are taken into account. Also the feedback report indicates respondent's position with respect to other respondents.

There may be four risk levels, i.e., high, medium, low, no risk, under which the group-specific indices fall. For example, when the user defines his/her performance in each question of the group to be the lowest possible, it is interpreted that user's well-being in this group represents high risk to his/her brains in the long run. As opposite, when the user defines his/her performance in each question of the group to be the highest possible, it is interpreted that user's performance is good in that specific group.

The overall evaluation takes into account all the questions in each group, and generates an evaluation index based on principles of the group-specific index calculation. It is possible that a group-specific index in a certain group represents high risk, whereas a group-specific index in another group represents low risk. The evaluation index takes into account the variations between group-specific indices.

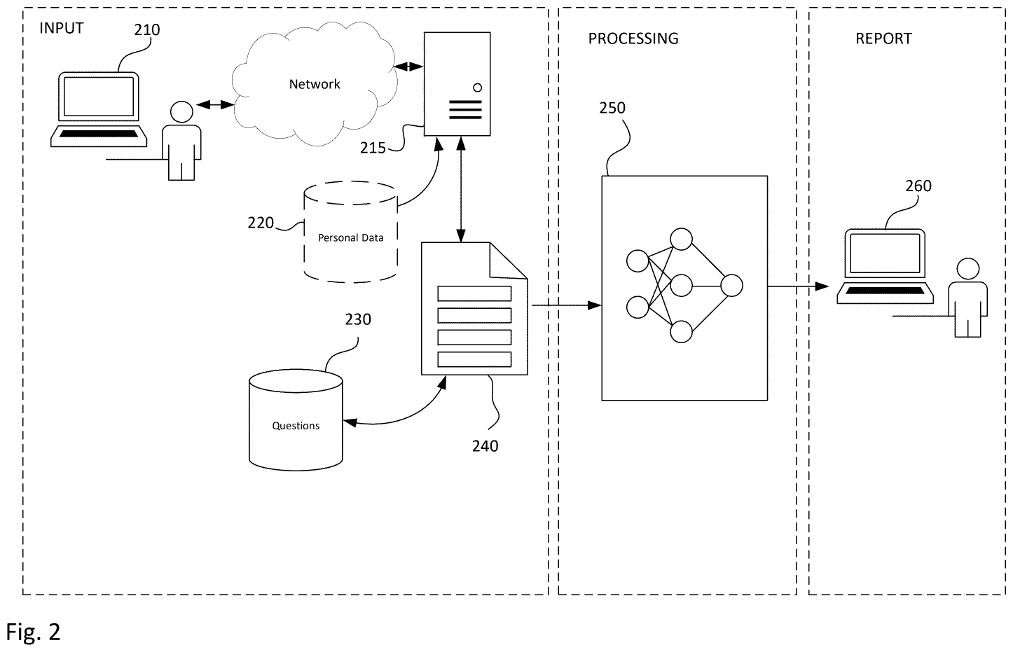

FIG. 2 illustrates a high-level architecture for a system according to an embodiment. As shown in FIG. 2, the system has been divided into three operation levels: input, processing, report. Elements that are operationally connected to the system are a user device 210, a network, a server 215, a database 330 and a query generation module 240. Optionally, the system may also be operationally connected to a personal information database 220.

The database 230 and the query generation module 240 may be stored on a server 215 of a (e.g., health-case) service provider, or may be stored on a server of a third-party and connected by the server 215. The user accesses the server 215 by his/her electronic device 210 through an identification module (not shown in the figure). During the identification process, personal data may be obtained from a database 220. Alternatively, the user may input the personal data during the identification process by themself. The access to the server 215 is implemented through a network, which can be in the form of wireless or wired network. The server provides a web site to the display of the electronic device 210, the web site comprising at least the questionnaire comprising questions according to the present solution. The questions have been selected by the query generation module 240. The query generation module 240 obtains query related data, i.e., questions, from a database 230 and modifies the questions according to personal data of the user. The query generation module 240 forms a survey form, which is displayed on a browser of the user's electronic device 210 through the server 215. The operation of the query generation module 240 may be based on machine learning, or other solution enabling artificial intelligence.

The respondent uses the browser to answer the questions, and the answers are provided to the processing unit for analysis. An algorithm 250 may be used to analyse data, and to determine the overall evaluation index and alternatively also the group-specific indices. In some examples, instead of an algorithm 250, the query generation module 240 may be configured to determine the group-specific indices and/or the overall index. The operation of the algorithm 250 may be based on machine learning, or other solution enabling artificial intelligence.

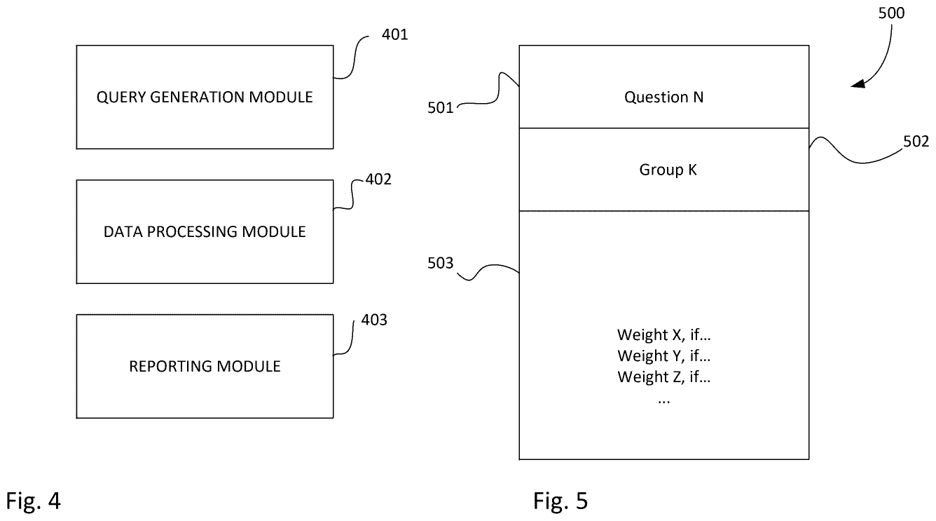

In the following, a short description on the computer program enabling the artificial intelligence is given. The computer program can comprise one or more algorithms, i.e., modules (for example the ones shown in FIG. 4), to implement various methods of the present embodiments. For example, the computer program may comprise the query generation module 401 for generating the query, i.e., for selecting suitable questions for a certain person and for determining weights for the selected question according to person's personal information and answers being given to one or more previous queries. For example, a retired person is not asked questions related to work.

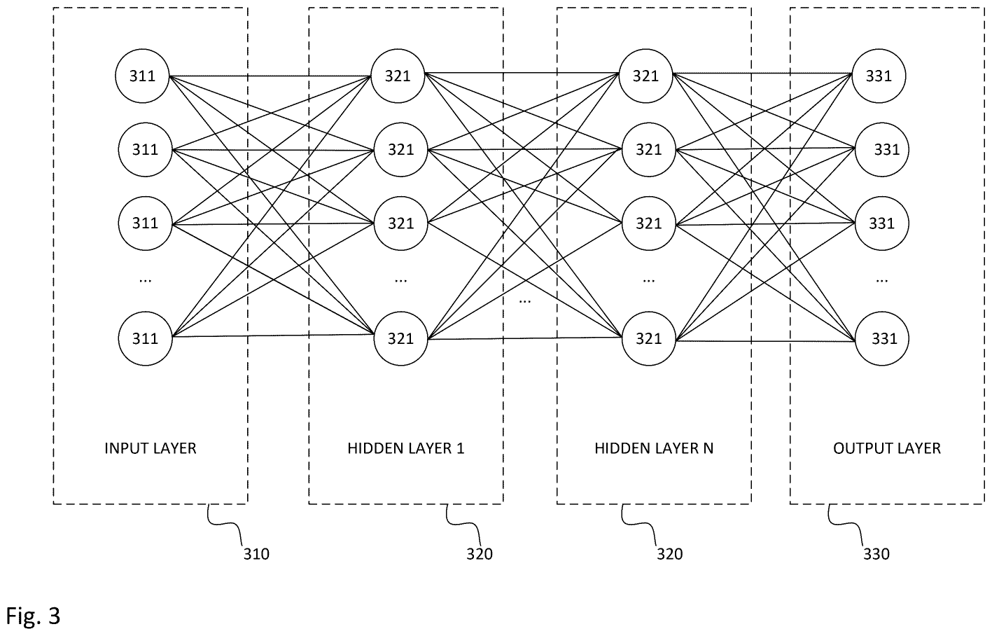

FIG. 3 illustrates an example of a neural network that can be used by the query generation module for generating the query. The neural network comprises an input layer 310, one or more hidden layers 320, and an output layer 330. The nodes 311 of the input layer 310 receives personal information of the user (i.e., the person answering to the query) and/or the answers from the earlier queries. Based on the personal information, the nodes 321 of the one or more hidden layers 320 determine a suitable set of questions for the user. The nodes 331 of the output layer 330 output the determined set of questions. At the same time, the nodes 321 of the one or more hidden layers 320 are able to determine a weight for each question. The determined weight can be based on the personal information of the user as well as answers given by the user in previous queries and/or for previous questions in the current query.

In addition, the computer program may comprise the data processing module 402 for determining the evaluation index and optionally also the group-specific indices. Each question of the query affects to an index of each group and to the evaluation index. The evaluation index may be formed by using the group-specific indices or by using each individual question. The indices take into account the weights defined for the questions, which weights are determined by the personal information of the respondent. The data processing module 420 may also gather the reference data, and obtain reference data for specific respondent, which reference data may comprise performance indices determined for other people and/or performance indices determined for the respondent in previous queries. In addition, the data processing module 420 may produce output based on the determined indices for the respondent. The data processing module 420 may thus be divided into several sub-modules, e.g., index and/or weight calculation sub-module; feedback generation sub-module; reference group gathering module.

The structure of the neural network shown in FIG. 3 is applicable also for data processing module. When used for analysis, the nodes 311 of the input layer 310 receives answers of the user (i.e., the person answering to the query) as input, as well as the weights determined for each question. Based on the input, the nodes 321 of the one or more hidden layers 320 determine the desired indices (e.g., group-specific indices, the evaluation index). The nodes 331 of the output layer 330 output the determined group-specific indices as well as the evaluation index.

The computer program may comprise also a module 403 for generating an individual feedback report based on the determined indices, and by taking into account single deviating answers when compared to answers given for earlier queries and to answers given by reference group. The reporting module 430 operates with the data processing module, and generates reports based on the results gathered and/or the feedback generated by the data processing module 420. The reports may be in electronic form, e.g. as a text-based report, or as an audio, video, or image file. The reporting module 430 outputs the reports, and delivers them to respective recipients (e.g., the user, the health-care provider, . . . ).

The modules shown in FIG. 4 can be stored in a memory of one or more physical device participating to the overall operation. For example, a server device (e.g., element 215 in FIG. 2) can comprise a processor and a memory, wherein the memory stores a computer program product having one or more of the computer modules. As another example, the query generator module can be part of an external query database, that is accessed by the system (or a server 215).

As said, the modules 401, 402, 403 may be based on machine learning or on neural network solution enabling artificial intelligence. Such solutions may be trained with data that has been (or is continuously) gathered from accomplished queries and received answers. In addition, the training data may originate from empirical studies and researches, and examinations carried out by medical or health-care service providers. Instead of training, the modules may be programmed in a fixed manner to operate according to a specified model.

The results that are obtained from the data processing module are delivered to a reporting module. The reporting module gathers the information received from the processing, and generates a feedback report to be delivered to the user and/or healthcare service provider. The feedback report comprises general view of user's considerations of their own situation. These results can reflect to potential brain and memory health risks. The feedback delivered to the health-care service provider may also contain an overall picture of the situation of the population or a client group. This feedback may also contain estimate of the situation of the brain and memory health situation in respective target group.

The results of a person are also delivered to a data storage gathering all the results of all respondents. Based on the gathered results, data concerning reference groups can be formed.

In addition, the results of a person can be used to specify person's personal activation program. The activity program may concentrate on fields where the user has the weakest results. By repeating the query time after time as part of the activation program, a graph will be resulted, which may indicate a decline or an ascent of the measured feature, e.g., the mind.

FIG. 5 illustrates an example of a data structure for a question (i.e., question data structure) being created according to an embodiment. In this example, the data structure comprises fields 501, 502, 503, wherein field 501 indicates the question, field 502 comprises information on the group which the question belongs to, and field 503 comprises other information, for example how the weights should be applied to the given answer. Each piece of question-related data can be stored in respective fields of the question data structure and can be modifiable. The system also comprises means for storing said question data structure.

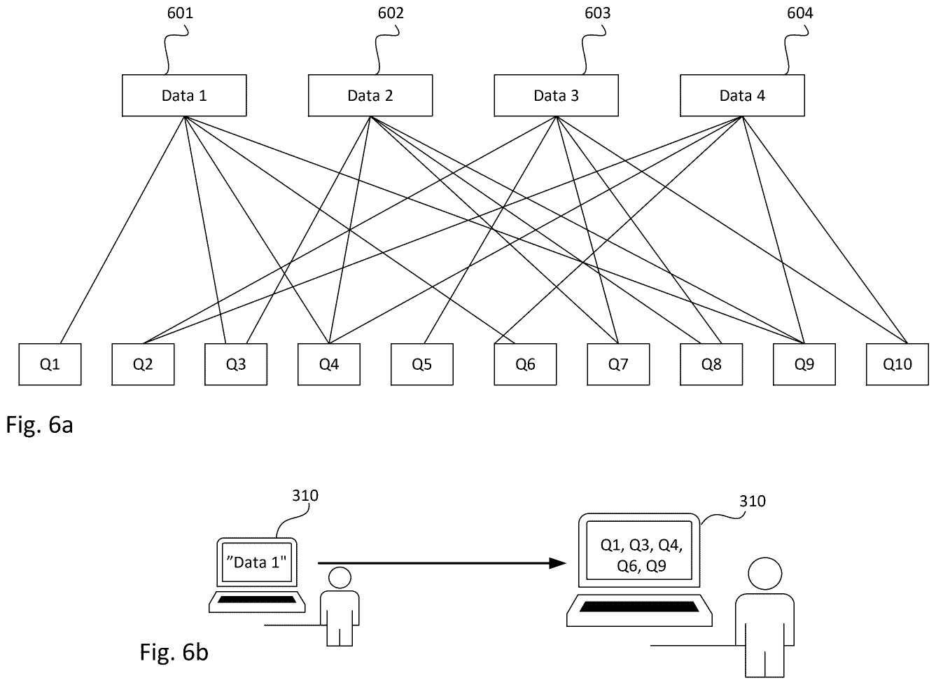

The questions may be stored in the question database in a tree-like hierarchy, where the upper-most parent node defines the set of questions to be asked. For example, if the background information states that the respondent is unemployed, questions related to work are ignored. This idea is illustrated in FIG. 6a in a simplified manner. Elements 601, 602, 603, 604 represent various background data, which control which questions from the set of questions Q1-Q10 are being selected to be presented to the respondent. For example, as illustrated in FIG. 6b, based on Data 1 601 being provided by the user device 310, the generated question set to be provided to the respondent comprises questions Q1, Q3, Q4, Q6, Q9. As another example, based on Data 3 603, the generated question set comprises questions Q2, Q5,Q7, Q8, Q10. It is to be noticed that the generated question set may dynamically evolve. For example, if a person answers “3” to question Q3, the query generation module may insert Q5 to the question set, even though Q5was not in the default set of question when the starting point was Data 1 601.

According to another embodiment, the questions may be stored in a large question pole in the database, where each question has a priority value according to which questions are selected for the user. For example, the query may be formed of 25 questions having the greatest priority. However, the questions may be dynamically changed according to the obtained personal information, whereupon questions having a lower priority by default may replace some of the questions having the higher priority in the query.

The data that controls the selection of the questions may also comprise the answers of the previous one or more queries. In the selection, questions improving the weakest field of performance may be emphasized, while the number of questions that are related to performance, where the respondent has succeeded, may be reduced. As an example, if a respondent is in good physical condition, but feels himself/herself lonely, questions to be asked may mainly relate to social activity and ways to improve the social activity, and such questions may be asked partially instead of questions relating to physical activity.

The system may also continuously track the development of a certain field of performance based on previous queries. If the development decreases, such a situation may be intervened by adding questions that may improve that field. Similarly, when the development is improved, the number of questions on that field may be reduced.

In previous, the operation of the system has been discussed from a perspective of one person. It is to be appreciated that the survey can be made to groups of people when such groups of people are being screened. The screening reveals what is the cognitive performance of people in some geographical area, in some age group, in some gender group, etc. The solution is not a diagnostic solution but aims to evaluate risk factors for cognitive performance in preventative manner.

An apparatus according to an embodiment comprises means for receiving data concerning user's personal information; means for determining at least one group of questions based on the received data, wherein the group of questions comprises psychological questions relating to cognitive performance of a user; means for determining weights for each question in said at least one group of questions based on the personal information; means for providing said at least one group of questions to a user; means for receiving an input from the user, said input comprises answers to the group of questions; and means for determining an evaluation index based on the answers received to all questions of said at least one group for questions, the evaluation index indicating user's cognitive performance. The means comprises at least one processor, and a memory including a computer program code, wherein the processor may further comprise processor circuitry. The memory and the computer program code are configured to, with the at least one processor, cause the apparatus to perform the method according to various embodiments.

The present embodiments provide advantages. For example, the method is easily deliverable to mass of people, whereupon the query can screen the status of the cognitive performance within a group of people. Since, the cognitive performance can be evaluated according to a set of psychological questions, there is no need to perform any measuring, but the respondent is able to carry out the query whenever needed. The evaluation method performs a risk analysis for any respondent, whereupon the system may guide the respondent to an activation model/program if needed. The activation model/program may be dynamically created according to the identified problems the respondent may have.

If desired, the different functions discussed herein may be performed in a different order and/or concurrently with other. Furthermore, if desired, one or more of the above-described functions and embodiments may be optional or may be combined.

Although various aspects of the embodiments are set out in the independent claims, other aspects comprise other combinations of features from the described embodiments and/or the dependent claims with the features of the independent claims, and not solely the combinations explicitly set out in the claims.

It is also noted herein that while the above describes example embodiments, these descriptions should not be viewed in a limiting sense. Rather, there are several variations and modifications, which may be made without departing from the scope of the present disclosure as, defined in the appended claims.

1. A method, comprising:
allowing an access of a user to an electronic service;
receiving data concerning user's personal information;
determining at least one group of questions from a question database based on the received data, wherein the group of questions comprises psychological questions relating to cognitive performance evaluation and wherein each question has one or more pre-defined weight options;
determining an applicable weight from said one or more pre-defined weight options for each question in said at least one group of questions based on the personal information;
providing said at least one group of questions to a user;
receiving an input from the user, said input comprises answers to the group of questions; and
determining an evaluation index based on the answers received to all questions of said at least one group for questions and based on the applicable weights for each question, the evaluation index indicating user's cognitive performance.

2. A method according to claim 1, further comprising determining group-specific index for each group of questions based on the answers received to such group, wherein the group-specific index takes into account the weights of each question.

3. The method according to claim 1, further comprising comparing the evaluation index to a reference group and optionally also to previous evaluation index/indices of the user.

4. The method according to claim 1, further comprising generating a feedback based on the group-specific index/indices, the evaluation index and result of the comparison.

5. The method according to claim 1, further comprising generating a question data structure, said structure comprises at least a question field and a weight field; and storing said question data structure in a database.

6. An apparatus comprising at least one processor, and a memory including a computer program code, wherein the memory and the computer program code are configured to, with the at least one processor, cause the apparatus at least to:
allow an access of a user to an electronic service;
receive data concerning user's personal information;
determine at least one group of questions from a question database based on the received data, wherein the group of questions comprises psychological questions relating to cognitive performance evaluation and wherein each question has one or more pre-defined weight options;
determine an applicable weight from said one or more pre-defined weight options for each question in said at least one group of questions based on the personal information;
provide said at least one group of questions to a user;
receive an input from the user, said input comprises answers to the group of questions; and
determine an evaluation index based on the answers received to all questions of said at least one group for questions and based on the applicable weights for each question, the evaluation index indicating user's cognitive performance.

7. The apparatus according to claim 6, further comprising computer program code to cause the apparatus to determine a group-specific index for each group of questions based on the answers received to such group, wherein the group-specific index takes into account the weights of each question.

8. The apparatus according to claim 6, further comprising computer program code to cause the apparatus to comparing compare the evaluation index to a reference group and optionally also to previous evaluation index/indices of the user.

9. The apparatus according to claim 6, further comprising computer program code to cause the apparatus to means for generating-generate a feedback based on the group-specific index/indices, the evaluation index and result of the comparison.

10. The apparatus according to claim 6, further comprising computer program code to cause the apparatus to generate a question data structure, said structure comprises at least a question field and a weight field; and to store said question data structure in a database.

11. A computer program product comprising computer program code configured to, when executed on at least one processor, cause an apparatus or a system to:
allow an access of a user to an electronic service;
receive data concerning user's personal information;
determine at least one group of questions from a question database based on the received data, wherein the group of questions comprises psychological questions relating to cognitive performance evaluation and wherein each question has one or more pre-defined weight options;
-determine an applicable weight from said one or more pre-defined weight options for each question in said at least one group of questions based on the personal information;
provide said at least one group of questions to a user;
receive an input from the user, said input comprises answers to the group of questions; and
determine an evaluation index based on the answers received to all questions of said at least one group for questions and based on the applicable weights for each question, the evaluation index indicating user's cognitive performance.
